(19) United States
(12) Patent Application Publication
(10) Pub. No.: US 2004/0033203 A1
(43) Pub. Date: Feb. 19, 2004

(54) Two-compartment device comprising at least one amphiphilic linear block polymer

(75) Inventors: Claude Dubief, Le Chesnay (FR); Franck Giroud, Clichy (FR); Isabelle Rollat-Corvol, Paris (FR)

Correspondence Address: LERNER, DAVID, LITTENBERG,, KRUMHOLZ & MENTLIK, 600 SOUTH AVENUE WEST, WESTFIELD, NJ, 07090, US

(73) Assignee: L'OREAL, Paris (FR)

(21) Appl. No.: 10/448,186

(22) Filed: May 29, 2003

Related U.S. Application Data
(60) Provisional application No. 60/387,423, filed on Jun. 11, 2002.

(30) Foreign Application Priority Data
May 31, 2002 (FR) 0206737

(51) Int. Cl. A61K 9/00

(52) U.S. Cl. 424/47

(57) ABSTRACT

The present invention relates to a two-compartment aerosol device comprising:

in a first compartment, a styling composition comprising at least one linear block copolymer comprising at least one hydrophilic block and at least one hydrophobic block, the hydrophilic block(s) representing at least 30% by weight of the linear block copolymer, with the exclusion of block copolymers of ethylene oxide and of propylene oxide, block copolymers containing urethane units and block copolymers containing siloxane units, and

in a second compartment, a compressed gas.

The present invention relates to a two-compartment aerosol device comprising a styling composition containing at least one amphiphilic block polymer and a compressed gas, and to a styling process.

Styling compositions such as lacquers and sprays, packaged in aerosol spray form, are generally composed of a liquid phase comprising at least one film-forming polymer in an alcoholic or aqueous-alcoholic cosmetically acceptable medium, and a propellant, which is a liquefied gas under reduced pressure or dissolved in the liquid phase.

In the field of hair products, it is desired to manufacture aerosol lacquers comprising no volatile organic compound such as ethanol or dimethyl ether, for essentially ecological reasons, while at the same time maintaining good hairstyle shaping and hold properties.

Document U.S. Pat. No. 5,626,840 describes hair fixing compositions that may be in aerosol form. They comprise a polyurethane in an aqueous or aqueous-alcoholic medium, a mineral or organic base and a solvent which may be water or a mixture of water and a polar organic solvent.

The said document describes a marked decrease in volatile organic compounds in the styling compositions.

The Applicant has discovered, surprisingly, that by using a two-compartment device comprising, in a first compartment, a styling composition containing at least one linear block copolymer comprising at least one hydrophilic block and at least one hydrophobic block, the hydrophilic block(s) representing at least 30% by weight of the linear block copolymer, with the exclusion of block copolymers of ethylene oxide and of propylene oxide, block copolymers containing urethane units and block copolymers containing siloxane units, and, in a second compartment, a compressed gas, it is possible to obtain a lacquer that has shaping and hold properties that are at the very least equivalent to those of the lacquers of the prior art, and which in addition comprises no volatile organic compound.

The said properties are obtained in particular when the styling composition comprises at least one linear block copolymer comprising at least one hydrophilic block and at least one hydrophobic block, the hydrophilic block(s) representing at least 30% by weight of the linear block copolymer, with the exclusion of block copolymers of ethylene oxide and of propylene oxide, block copolymers containing urethane units and block copolymers containing siloxane units.

One subject of the present invention is thus a two-compartment aerosol device comprising

in a first compartment, a styling composition comprising at least one linear block copolymer comprising at least one hydrophilic block and at least one hydrophobic block, the hydrophilic block(s) representing at least 30% by weight of the linear block copolymer, with the exclusion of block copolymers of ethylene oxide and of propylene oxide, block copolymers containing urethane units and block copolymers containing siloxane units, and

in a second compartment, a compressed gas.

Another subject of the present invention consists of a styling process using the device of the invention.

Other subjects, characteristics, aspects and advantages of the invention will emerge even more clearly on reading the description and the various examples that follow.

Preferably, the compressed gas is chosen from air, nitrogen and carbon dioxide, and mixtures thereof, air being particularly preferred.

The said compressed gas is preferably used at a pressure of between 1 and 12 bar and better still between 9 and 11 bar.

The linear block copolymers that may be used according to the present invention are “amphiphilic” copolymers, i.e. copolymers comprising both hydrophobic blocks and hydrophilic blocks.

According to the present invention, the term “hydrophobic blocks” means blocks comprising at least 75 mol % of water-insoluble monomers, and the term “hydrophilic blocks” means blocks comprising at least 75 mol % of water-soluble monomers.

The water-soluble monomers forming the hydrophilic blocks of the block copolymers used in the present invention may be of anionic, nonionic or cationic nature and may be used alone or in the form of a mixture containing two or more different monomers.

Examples of anionic water-soluble monomers that may be mentioned include ethylenically unsaturated carboxylic acids, such as acrylic acid, methacrylic acid, itaconic acid, fumaric acid, crotonic acid, maleic acid, 2-acrylamido-2-methylpropanesulphonic acid, styrenesulphonic acid, vinylsulphonic acid and vinylphosphonic acid.

The nonionic water-soluble monomers include, inter alia, acrylamide, C₁₋₆ N-alkyl or C₁₋₃ N,N-dialkyl acrylamides, polyethylene glycol acrylate, polyethylene glycol methacrylate, N-vinylacetamide, N-methyl-N-vinylacetamide, N-vinylformamide, N-methyl-N-vinylformamide, N-vinyllactams comprising a cyclic group of 4 to 9 carbon atoms, vinyl alcohol (copolymerized in the form of vinyl acetate and then hydrolysed), ethylene oxide, hydroxyethyl acrylate, hydroxypropyl acrylate, hydroxyethyl methacrylate and hydroxypropyl methacrylate.

Finally, the cationic water-soluble monomers include, for example, dimethyldiallylammonium chloride, methylvinylimidazolium chloride, 2-vinylpyridine, 4-vinylpyridine, 2-methyl-5-vinylpyridine, vinylamine, the monomers of formula

H₂C═CR₁—CO—X₂

in which

R₁ represents a hydrogen atom or a methyl group,

X₂ represents a linear or branched C₁₋₆ hydrocarbon-based group bearing at least one primary, secondary or tertiary amine function or at least one quaternary nitrogen atom, or a group of formula NHR₂ or of formula NR₂R₃ in which R₂ and R₃ represent, independently of each other, a linear or branched C₁₋₆ hydrocarbon-based group bearing at least one primary, secondary or tertiary amine function or at least one quaternary nitrogen atom.

The water-insoluble monomers forming the hydrophobic blocks of the block copolymers are preferably chosen from vinylaromatic monomers such as styrene and its alkyl derivatives, for instance 4-butylstyrene, α-methylstyrene and vinyltoluene, dienes such as butadiene and 1,3-hexadiene, and alkyl derivatives of dienes such as isoprene and dimethylbutadiene, chloroprene, C₁₋₁₀ alkyl, C₆₋₁₀ aryl or C₆₋₁₀ aralkyl acrylates and C₁₋₁₀ alkyl, C₆₋₁₀ aryl, or C₆₋₁₀ aralkyl methacrylates, for instance methyl, ethyl, n-butyl, 2-ethylhexyl, tert-butyl, isobornyl, phenyl or benzyl (meth)acrylates, vinyl acetate, vinyl ethers of formula CH₂═CH—O—R and allyl ethers of formula CH₂═CH—CH₂—O—R in which R represents a C₁₋₆ alkyl group, acrylonitrile, vinyl chloride, vinylidene chloride, caprolactone, ethylene, propylene, vinyl monomers that are fluorinated or that contain a perfluoro chain, such as fluoroalkyl acrylates and methacrylates or alkyl fluoroacrylates.

As indicated above as regards the definition of the hydrophobic and hydrophilic blocks of the block copolymers, the water-insoluble monomers and the water-soluble monomers represent at least 75 mol %, respectively, of the hydrophobic and hydrophilic blocks. In other words, each hydrophobic block may comprise up to 25 mol % of one or more water-soluble monomers. This proportion is preferably not more than 10 mol % and ideally less than or equal to 5 mol %.

Similarly, each hydrophilic block may comprise up to 25 mol %, preferably up to 10 mol % and ideally up to 5 mol % of one or more water-insoluble monomers.

The linear block copolymers used also obviously include those in which the hydrophilic blocks and the hydrophobic blocks consist exclusively of water-soluble monomers and of water-insoluble monomers, respectively. These blocks may be homopolymer blocks or copolymer blocks containing two or more than two different monomers of the same type.

Preferably, the linear block copolymers of the invention are water-soluble or water-dispersible, and even more preferably they are water-soluble.

The term “water-soluble monomer or polymer” means a monomer or polymer which, when introduced into water at a temperature of 25° C. and at a weight concentration equal to 0.5%, produces a macroscopically homogeneous and transparent solution, i.e. a solution with a light transmittance value, at a wavelength equal to 500 nm, through a sample 1 cm thick, of at least 70% and preferably of at least 80%.

The water-dispersible copolymers according to the invention may be in the form of lattices or pseudolattices.

Preferably, the linear block copolymers according to the invention are water-soluble.

This ability to be dissolved or finely dispersed in water is linked to the large proportion of hydrophilic blocks in the amphiphilic block copolymers.

This proportion must be at least 30% by weight and is preferably greater than or equal to 60% by weight, without, however, exceeding 97% by weight and preferably 95% by weight.

The number-average molecular mass of each block, whether it is hydrophobic or hydrophilic, a copolymer or a homopolymer, is preferably between 500 and 100 000 and in particular between 500 and 50 000, with a polydispersity index (M_{w}/Mₙ) of between 1.01 and 3.0 and preferably between 1.1 and 2.5.

The linear block copolymers used in the present invention may be

diblock copolymers of formula AB,

triblock copolymers of formula ABA or BAB, and

multiblock copolymers comprising at least two hydrophilic blocks and at least two hydrophobic blocks arranged alternately, each A representing a hydrophilic block and each B representing a hydrophobic block, the blocks A of the same polymer possibly being identical or different and the blocks B of the same polymer possibly being identical or different.

Diblock copolymers and triblock copolymers comprising a hydrophilic central block and two hydrophobic side blocks are particularly preferred.

The block polymers of the invention may be prepared by the synthetic processes conventionally used for obtaining block polymers. Mention may be made, for example, of anionic or cationic polymerization and controlled free-radical polymerization (see “New Method of Polymer Synthesis”, Blackie Academic & Professional, London, 1995, Volume 2, page 1, or Trends Polym. Sci. 4, page 183 (1996) by C. J. Hawker), which may be used according to different processes, for instance atom transfer radical polymerization (ATRP) (see JACS, 117, page 5614 (1995) from Matyjasezwski et al.), and the method using radicals such as nitroxides (Georges et al., Macromolecules, 1993, 26, 2987).

These processes may also be used to obtain just one of the two types of blocks of the polymer of the invention, the other block being introduced into the final polymer by means of the initiator used or alternatively via a coupling reaction between the hydrophilic and hydrophobic blocks.

The amount of amphiphilic linear block copolymers in the compositions of the present invention depends on a large number of parameters, among which mention may be made of the molecular mass of the copolymers, the number and size of the hydrophilic and hydrophobic blocks, the amount of polymers that are beneficial to the hair, and especially the viscosity of the composition that it is desired to obtain.

Satisfactory results are generally obtained with an amount of linear block copolymers of between 0.01% and 10% by weight and preferably between 0.1% and 5% by weight relative to the weight of the hair composition.

The styling composition contained in the device according to the invention advantageously has a water content of between 30% and 99.9% by weight, preferably between 50% and 99% by weight and even more preferably between 75% and 98% by weight relative to the total weight of the said composition.

The styling composition may also comprise additives such as silicones in soluble, dispersed or microdispersed form, treating active agents, moisturizers, for instance glycerol, UV-screening agents, acids, bases, plasticizers, solubilizers, preserving agents, vitamins and provitamins, colourants, pigments, anionic, cationic, nonionic or amphoteric surfactants, fragrances and anti-corrosion agents, and mixtures thereof.

A person skilled in the art will take care to select the optional additives and the amount thereof such that they do not harm the properties of the compositions of the present invention.

These additives are especially present in the composition according to the invention in an amount ranging from 0 to 20% by weight relative to the total weight of the composition.

Preferably, the two-compartment aerosol device consists of an outer aerosol can comprising an inner pocket hermetically welded to a valve. The composition is introduced into the inner pocket and a compressed gas is introduced between the pocket and the can at a pressure that is sufficient to eject the product in the form of a spray through the orifice of a nozzle. Such a device is sold under the name EP Spray by the company EP Spray System SA.

The aerosol devices of the invention are preferably hair lacquers.

The present invention also relates to a styling process which consists in vaporizing the styling composition contained in the aerosol device according to the invention onto wet or dry hair.

The examples that follow are given as illustrations of the present invention.

### EXAMPLE 1

A styling composition was prepared using the ingredients below:

| | % by weight |
|---|---|
| ⁽¹⁾ Cationic block copolymer formed from | 5 AM* |
| a polystyrene block and a poly (N-methyl- | |
| N-vinylpyridinium iodide) block | |
| Demineralized water | qs 100 |

*Active material

The composition prepared above was introduced into an aerosol distribution device sold under the name EP Spray by the company EP Spray System S.A. described above. A valve of reference 6001 format D6 is attached to a standard aerosol can and the diffuser is a vortex-nozzle diffuser.

The pocket is filled with the composition as indicated above. Compressed air is introduced between the pocket and the can.

The composition was vaporized onto dry hair. The spraying takes place in the form of a gentle spray.

After drying, a head of hair with good hold is obtained.

1. Two-compartment aerosol device comprising:
in a first compartment, a styling composition comprising at least one linear block copolymer comprising at least one hydrophilic block and at least one hydrophobic block, the hydrophilic block(s) representing at least 30% by weight of the linear block copolymer, with the exclusion of block copolymers of ethylene oxide and of propylene oxide, block copolymers containing urethane units and block copolymers containing siloxane units, and
in a second compartment, a compressed gas.

2. Aerosol device according to claim 1, characterized in that the compressed gas is chosen from air, nitrogen and carbon dioxide, and mixtures thereof, air being particularly preferred.

3. Aerosol device according to claim 1 or 2, characterized in that the pressure of the compressed gas is between 1 and 12 bar.

4. Aerosol device according to claim 3, characterized in that the pressure of the compressed gas is between 9 and 11 bar.

5. Aerosol device according to one of the preceding claims, characterized in that the linear block copolymer is chosen from diblock copolymers of formula AB, triblock copolymers of formula ABA or BAB and multiblock copolymers comprising at least two hydrophilic blocks and at least two hydrophobic blocks arranged alternately, each A representing a hydrophilic block and each B representing a hydrophobic block, the blocks A of the same polymer possibly being identical or different and the blocks B of the same polymer possibly being identical or different.

6. Aerosol device according to claim 5, characterized in that the linear block copolymer is chosen from diblock copolymers and triblock copolymers comprising a hydrophilic central block and two hydrophobic side blocks.

7. Aerosol device according to any one of the preceding claims, characterized in that the hydrophilic blocks are formed from water-soluble monomers chosen from anionic water-soluble monomers, nonionic water-soluble monomers and cationic water-soluble monomers or a mixture thereof.

8. Aerosol device according to claim 7, characterized in that the anionic water-soluble monomers are chosen from ethylenically unsaturated carboxylic acids, 2-acrylamido-2-methylpropanesulphonic acid, styrenesulphonic acid, vinylsulphonic acid and vinylphosphonic acid.

9. Aerosol device according to claim 7, characterized in that the nonionic water-soluble monomers are chosen from acrylamide, C₁₋₆ N-alkyl or C₁₋₃ N,N-dialkyl acrylamides, polyethylene glycol acrylate, polyethylene glycol methacrylate, N-vinylacetamide, N-methyl-N-vinylacetamide, N-vinylformamide, N-methyl-N-vinylformamide, N-vinyllactams comprising a cyclic group of 4 to 9 carbon atoms, vinyl alcohol, ethylene oxide, hydroxyethyl acrylate, hydroxypropyl acrylate, hydroxyethyl methacrylate and hydroxypropyl methacrylate.

10. Aerosol device according to claim 7, characterized in that the cationic water-soluble monomers are chosen from dimethyldiallylammonium chloride, methylvinylimidazolium chloride, 2-vinylpyridine, 4-vinylpyridine, 2-methyl-5-vinylpyridine, vinylamine, the monomers of formula
H₂C═CR₁—CO—X₂
in which
R₁ represents a hydrogen atom or a methyl group,
X₂ represents a linear or branched C₁₋₆ hydrocarbon-based group bearing at least one primary, secondary or tertiary amine function or at least one quaternary nitrogen atom, or a group of formula NHR₂ or of formula NR₂R₃ in which R₂ and R₃ represent, independently of each other, a linear or branched C1-6 hydrocarbon-based group bearing at least one primary, secondary or tertiary amine function or at least one quaternary nitrogen atom.

11. Aerosol device according to any one of the preceding claims, characterized in that the hydrophobic blocks are formed from water-insoluble monomers chosen from vinylaromatic monomers, dienes and alkyl derivatives of dienes, chloroprene, C₁₋₁₀ alkyl, C₆₋₁₀ aryl or C₁₋₁₀ aralkyl acrylates, C₁₋₁₀ alkyl, C₆₋₁₀ aryl or C₁₋₁₀ aralkyl methacrylates, vinyl acetate, vinyl ethers of formula CH₂═CH—O—R and allyl ethers of formula CH₂═CH—CH₂—O—R in which R represents a C₁₋₆ alkyl group, acrylonitrile, vinyl chloride, vinylidene chloride, caprolactone, ethylene, propylene and vinyl monomers that are fluorinated or that contain a perfluoro chain.

12. Aerosol device according to any one of claims 7 to 10, characterized in that the hydrophilic blocks contain up to 25 mol %, preferably up to 10 mol % and ideally up to 5 mol % of one or more water-insoluble monomers according to claim 11.

13. Aerosol device according to claim 11, characterized in that the hydrophobic block(s) contain(s) up to 25 mol %, preferably up to 10 mol % and ideally up to 5 mol % of one or more water-soluble monomers according to any one of claims 8 to 10.

14. Aerosol device according to any one of the preceding claims, characterized in that the block copolymer(s) is (are) water-soluble or water-dispersible.

15. Aerosol device according to claim 14, characterized in that the block copolymer(s) is (are) water-soluble.

16. Aerosol device according to any one of the preceding claims, characterized in that the hydrophilic block(s) represent(s) at least 30% by weight of the linear block copolymer.

17. Hair composition according to any one of the preceding claims, characterized in that the linear block copolymer(s) is (are) present in a proportion of from 0.01% to 10% by weight and preferably in a proportion of from 0.1% to 5% by weight relative to the hair composition.

18. Aerosol device according to any one of the preceding claims, characterized in that the styling composition has a water content of between 30% and 99.9% by weight, preferably between 50% and 99% by weight and even more preferably between 75% and 98% relative to the total weight of the said composition.

19. Aerosol device according to any one of the preceding claims, characterized in that the styling composition also comprises additives chosen from silicones in soluble, dispersed or microdispersed form, treating active agents, moisturizers, UV-screening agents, acids, bases, plasticizers, solubilizers, preserving agents, vitamins and provitamins, colourants, pigments, anionic, cationic, nonionic or amphoteric surfactants, fragrances and anti-corrosion agents, and mixtures thereof.

20. Aerosol device according to any one of the preceding claims, characterized in that it constitutes a hair lacquer.

21. Styling process, characterized in that the styling composition contained in the aerosol device according to the invention is vaporized onto wet or dry hair.
